(19) United States
(12) Patent Application Publication
(10) Pub. No.: US 2018/0035702 A1
(43) Pub. Date: Feb. 8, 2018

(54) GLUCOSYLATED STEVIOL GLYCOSIDE COMPOSITION AS A FLAVOR MODIFIER

(71) Applicant: PureCircle Sdn Bhd, Kuala Lumpur (MY)

(72) Inventors: Siddhartha PURKAYASTHA, Chicago, IL (US); Karen GRENUS, Aurora, IL (US); John MARTIN, Chicago, IL (US)

(21) Appl. No.: 15/625,365

(22) Filed: Jun. 16, 2017

Related U.S. Application Data
(63) Continuation-in-part of application No. 15/478,732, filed on Apr. 4, 2017.
Application No. 15/478,732 is a continuation of application No. 14/855,767, filed on Sep. 16, 2015, now abandoned.
Application No. 14/855,767 is a continuation of application No. 14/005,852, filed on Sep. 18, 2013, now abandoned.
Application No. 14/005,852 is a continuation-in-part of application No. PCT/US2011/033912, filed on Apr. 26, 2011.
(60) Provisional application No. 61/466,150, filed on Mar. 22, 2011.

(51) Int. Cl. C12P 19/18; A23L 2/60; A21D 2/18

(52) U.S. Cl. CPC A23L 27/30; C12P 19/18; A21D 2/181; A23L 2/60; A23L 27/88; A23L 27/36; A23L 27/33; A23V 2002/00

(57) ABSTRACT

A taste and flavor profile enhancing composition is described. The composition includes glucosylated steviol glycosides which can enhance the intensity of a taste and/or a flavor in a food or beverage product.

### BACKGROUND

Stevia rebaudiana Bertoni is a perennial shrub of the Asteraceae (Compositae) family native to certain regions of South America. The leaves of the plant contain from 10 to 20% of diterpene glycosides, which are around 150 to 450 times sweeter than sugar. The leaves have been traditionally used for hundreds of years in Paraguay and Brazil to sweeten local teas and medicines.

At present there are more than 230 Stevia species with significant sweetening properties. The plant has been successfully grown under a wide range of conditions from its native subtropics to the cold northern latitudes.

The extract of the Stevia rebaudiana plant contains a mixture of different sweet diterpene glycosides, which have a single base—steviol—and differ by the presence of carbohydrate residues at positions C13 and C19. These glycosides accumulate in Stevia leaves and compose approximately 10%-20% of the total dry weight. Typically, on a dry weight basis, the four major glycosides found in the leaves of Stevia are Dulcoside A, Rebaudioside C, Rebaudioside A and Stevioside. Other glycosides identified in Stevia extract include Rebaudioside B, D, E, and F, Steviolbioside and Rubusoside.

Steviol glycosides differ from each other by sweetness power as well as other sensory features contributing to taste quality such as bitterness, lingering aftertaste, etc. Kinghorn, A. D., Stevia: The genus Stevia, Taylor & Francis, London (2002). The taste quality of a steviol glycoside is one of the major characteristics which is decisive for its usage in various food and beverage applications. The sweetness and taste quality of stevia glycosides are set forth below in Table 1.

TABLE 1
Steviol Glycoside molecules and their sweetness and taste quality

| | | | Sweetening | |
|---|---|---|---|---|
| Steviol | | | power relative | Taste |
| Glycosides | R1 (C19) | R2 (C13) | to sucrose | Quality |
| Stevioside (G3) | β-Glc | β-Glc-β-Glc(2-1) | 150-300 | 0 |
| Rebaudioside A | β-Glc | β-Glc-β-Glc(2-1)- | 200-400 | +2 |
| (G4) | | β-Glc(3-1) | | |
| Rebaudioside B | H | β-Glc-β-Glc(2-1)- | 300-350^{a} | 0 |
| (G3) | | β-Glc(3-1) | | |
| Rebaudioside C | β-Glc | β-Glc-α-Rha(2-1)- | 50-120 | −1 |
| (G4) | | β-Glc(3-1) | | |
| Rebaudioside D | β-Glc-β-Glc(2-1) | β-Glc-β-Glc(2-1)- | 200-300 | +3 |
| (G5) | | β-Glc(3-1) | | |
| Rebaudioside E | β-Glc-β-Glc(2-1) | β-Glc-β-Glc(2-1) | 250-300 | +1 |
| (G4) | | | | |
| Rebaudioside F | β-Glc | β-Glc-β-Xyl(2-1)- | Na | Na |
| (G4) | | β-Glc(3-1) | | |
| Rubusoside (G2) | β-Glc | β-Glc | 110 | −2 |
| Steviolmonoside | H | β-Glc | Na | Na |
| (G1) | | | | |
| Steviolbioside | H | β-Glc-β-Glc(2-1) | 100-125 | −3 |
| (G2) | | | | |
| DulcosideA (G3) | β-Glc | β-Glc-α-Rha(2-1) | 50-120 | −2 |

Previous studies show a certain correlation between number of glycosidic residues and taste quality of a steviol glycoside. When comparing steviol glycosides, rebaudioside A (G4, having 4 glucose residues) clearly surpasses stevioside and rebaudioside B (G3, each having 3 glusose residues) in taste quality. Steviolbioside and rubusoside (G2, each having 2 glucose residues) have a taste quality that is proven to be significantly inferior to that of stevioside (G3). In addition, the taste quality of rhamnosylated glycosides is inferior compared to that of glucosylated glycosides. Tanaka, O., “Improvement of Taste of Natural Sweeteners,” Pure & Appl. Chem., Vol. 69, No. 4, pp. 675-683 (1997).

The sweetness and taste quality of steviol glycoside molecules containing only glucose units are plotted in FIGS. 1a-1b. Steviol glycosides with a greater number of glucose residues, for example, more than two glucose residues, show a better taste quality. Particularly mono- and di-glucosyl forms of stevioside (having 4 glucose residues (G4) and 5 glucose residues (G5) respectively) possess significantly better taste quality. Tanaka, O., “Improvement of Taste of Natural Sweeteners,” Pure & Appl. Chem., Vol. 69, No. 4, pp. 675-683 (1997).

It is known that the undesired taste attributes can be substantially reduced or eliminated by the reaction of intermolecular transglycosylation of various enzymes, upon which the attachment of new carbohydrates at positions C13 and C19 of steviol glycosides takes place. Tanaka (1997) evaluated the effect of adding glucose molecules to purified stevioside molecules by tranglycosylation. The resulting glucosylated steviosides were evaluated for their sweetness and taste quality. The improvement of taste quality was greater when the glucose units were added to the C19 position rather than at the C13 position.

Various enzymes were used to conduct such transglycosylation. Pullulanase, isomaltase (Lobov, S. V. et al., “Enzymic Production of Sweet Stevioside Derivatives: Transglucosylation by Glucosidases,” Agric. Biol. Chem., Vol. 55, No. 12, pp. 2959-2965 (1991)), β-galactosidase (Kitahata, S. et al., “Production of Rubusoside Derivatives by Transgalactosylation of Various β-Galactosidases,” Agric. Biol. Chem., Vol. 53, No. 11, pp. 2923-2928 (1989)), and dextrine saccharase (Yamamoto, K. et al., Biosci. Biotech. Biochem., Vol. 58, No. 9, pp. 1657-1661 (1994)) were used as enzymes with pullulan, maltose, lactose, and partially hydrolyzed starch being donors.

The transglucosylation of steviol glycosides was also achieved by the action of cyclodextrin glucanotransferases (CGTase). The obtained sweeteners possessed improved sweetness without bitterness and licorice taste (U.S. Pat. Nos. 4,219,571, 7,838,044, and 7,807,206).

With an increase in the number of glucose units in steviol glycoside molecules (for example, from stevioside to Rebaudioside A), the sweetness intensity increases and sweetness profile (taste) improves. However, the relative sweetness does not increase significantly beyond a certain level with a further increase of glucose units, as shown in FIG. 1a. The published data show that the sweetness quality improves with the addition of glucose units, but does not explicitly or implicitly mention that the addition of glucose units contributes to a reduction of sweetness.

### BRIEF SUMMARY OF THE INVENTION

The present invention is directed to a taste and flavor profile enhancing composition. The composition includes glucosylated steviol glycosides which can enhance the intensity of a taste and/or a flavor in a food or beverage product. In some embodiments, the glucosylated steviol glycosides may include a plurality of glucose units. For example, the glucosylated steviol glycosides may include three, four, five, or more than five glucose units.

The present invention is also directed to a food or beverage product having an intense taste and flavor profile, wherein the food or beverage product includes a taste and flavor enhancing composition comprising glucosylated steviol glycosides. A wide range of food and beverage products, such as, but not limited to, carbonated soft drinks, fruit juices, dairy foods, dairy beverages, baked goods, cereal products, and table top sweeteners, may be made in accordance with the present invention. The taste and flavor profile of a food or beverage product including a taste and flavor enhancing composition, wherein the taste and flavor enhancing composition includes glucosylated steviol glycosides, may be more intense than a comparative taste and flavor profile of a comparative food or beverage product which does not include the taste and flavor enhancing composition. Moreover, the mouthfeel of a food or beverage product including the taste and flavor enhancing composition, wherein the taste and flavor enhancing composition includes glucosylated steviol glycosides, may be improved in relation to a mouthfeel of a comparative food or beverage product which does not include the taste and flavor enhancing composition.

The present invention is further directed to a method of increasing the taste and flavor intensity of a food or beverage product, including the step of adding a taste and flavor enhancing composition to the food or beverage product, wherein the taste and flavor enhancing composition includes glucosylated steviol glycosides. The present invention is also directed to a method of improving the organoleptic properties of a food or beverage product including a high fructose syrup, including the step of adding the taste and flavor enhancing composition to the food or beverage product. For example, adding the taste and flavor enhancing composition may cause the high fructose syrup, such as high fructose corn syrup, to taste more like sugar. Also, if the high fructose syrup is high fructose corn syrup 42 (HFCS 42), adding the taste and flavor enhancing composition may cause the HFCS 42 to taste more like high fructose corn syrup 55 (HFCS 55).

The present invention is also directed to a method of making a taste and flavor enhancing composition, including: extracting steviol glycosides from leaves of a Steviol rebaudiana Bertoni plant, and transglycosylating the steviol glycosides to add glucose units to the steviol glycosides. In some embodiments, transglycosylating the steviol glycosides includes enzymatic transglycosylation using an enzyme. Examples of enzymes which may be used in accordance with the present invention include, but are not limited to, pullulanase, isomaltase, β-galactosidase, dextrine saccharase, and cyclodextrin glucotransferase.

The present invention is further directed to a method of making a food or beverage product, including: adding a taste and flavor enhancing composition including glucosylated steviol glycosides, and adding a reduced amount of erythritol, wherein the reduced amount of erythritol is less than the amount of erythritol in a comparative food or beverage composition which does not include the taste and flavor enhancing composition. The mouthfeel of the food or beverage product is similar to the mouthfeel of the comparative food or beverage product, even though the comparative food or beverage product contains a higher level of erythritol.

The foregoing has outlined rather broadly the features and technical advantages of the present invention in order that the detailed description of the invention that follows may be better understood. Additional features of the invention which form the subject of the claims of the invention will be described hereinafter. It should be appreciated by those skilled in the art that the specific embodiments disclosed may be readily utilized as a basis for modifying or designing other methods or structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description.

### BRIEF DESCRIPTION OF THE DRAWINGS

FIG. 1a is a bar graph showing the relative sweetness of steviol glycoside molecules with increasing numbers of glucose units. FIG. 1b is a bar graph showing the taste quality of steviol glycoside molecules with increasing numbers of glucose units.

FIG. 2 shows the chemical structures of stevioside and glucosyl stevioside.

FIG. 3 is a graph of the sweetness potency, or sucrose equivalent value (SEV), of stevia glycosides at a 5% sugar sweetness level.

FIG. 4a is a graph of the relative ratings of the sweetness intensity and sweet onset of mango-passion fruit flavored water beverages to which different stevia ingredients were added, and which did not include sugar. FIG. 4b is a graph of the relative ratings of the passion fruit flavor and mango flavor of these mango-passion fruit flavored water beverages.

FIG. 5a is a graph of the relative ratings of the sweetness intensity and sweet onset of mango-passion fruit flavored water beverages to which different stevia ingredients were added, and which included sugar. FIG. 5b is a graph of the relative ratings of the passion fruit flavor and mango flavor of these mango-passion fruit flavored water beverages.

FIG. 6 is a graph of the SEV (sucrose equivalent value) contribution of NSF-02 plotted against the concentration of NSF-02 in ppm, in acidified water and in acidified sugar solutions. NSF-02 (Natural Sweet Flavor #2) contains glucosylated steviol glycosides and dextrin, and is discussed further below.

FIG. 7 is a graph showing the synergy of NSF-02 with stevia exract, in a plot of the reduction of stevia extract against the concentration of NSF-02.

FIG. 8 is a bar graph showing the relative ratings of various attributes in an alcoholic beverage.

FIG. 9 is a bar graph showing the relative ratings of various attributes in a savory snack product.

FIG. 10 is a bar graph showing the relative ratings of various attributes in a fat-based dressing.

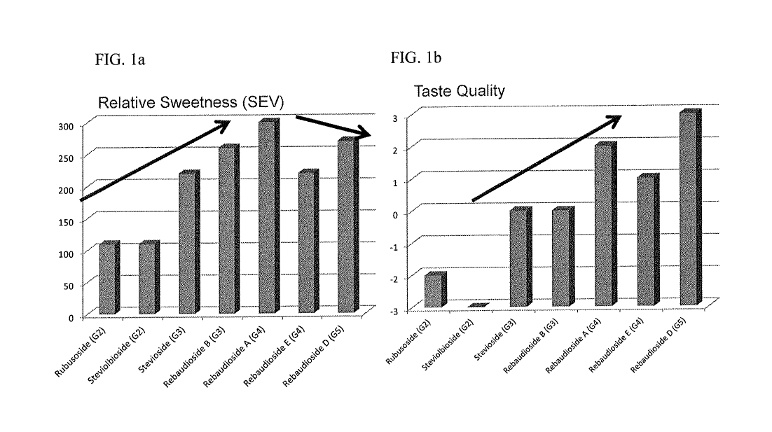

### DETAILED DESCRIPTION

It has been unexpectedly discovered that glucosylation of steviol glycosides beyond a certain number of glucose units effectively reduces sweetness. It has also been discovered that with the reduction of sweetness, the glucosylated steviol glycosides can contribute to the modification of flavor and sweetness profiles. Therefore, while sweetness decreases with glucosylation, flavor modification increases. The steviol glycosides mixture provides a certain amount of sweetness, but the present invention shows that the glucosylated steviol glycosides (hereinafter “GSG”) enhance the flavor and sweetness profile in a wide range of applications, such as those listed in, but not limited by, the categories shown below in Table 2.

TABLE 2
GSG Applications

| Application | Product | Results |
|---|---|---|
| Beverages | Apple Blueberry | Significantly more berry flavor intensity than the |
| | Juice Drink | Control. Directionally, more acceptable for overall |
| | | flavor. |
| | Orange Pineapple | Significantly more orange flavor intensity, pineapple |
| | Passion Fruit | flavor intensity, and sweetness intensity. |
| | Carbonated Drink | |
| Baked | Lemon Poppy | Significantly more overall flavor intensity and |
| Goods | Seed Muffins | sweetness intensity than the Control. |
| | | Directionally, the Test sample was more acceptable |
| | | overall and had more lemon flavor intensity. |
| Processed | Strawberry | Significantly less bitter than the control. |
| Fruits | Topping | |
| Dairy | Banana Flavored | Significantly more overall flavor intensity, banana |
| | Milk Drink | flavor intensity, sweetness intensity, tartness intensity |
| | | and bitterness intensity. |

Similar taste and flavor improvements were found in other categories of products, including, but not limited to, table top sweeteners, sauces and gravies, confectionery products, baked goods, cereals, snacks, and fruit and vegetable preparations.

### EXAMPLES

In the following examples, the percentages in the formulas refer to percentages by weight.

### Example 1

### Evaluation of Iso-Sweetness of Steviol Glycosides

To evaluate the iso-sweetness of steviol glycosides (SG) and glucosylated steviol glycosides (GSG), a series of samples were selected as shown below in Table 3. The GSG was produced by treating the raw materials, steviol glycosides extracted from the Stevia plant, and starch extracted from tapioca, with a natural enzyme. The enzyme transfers glucose units from starch to the steviol glycosides. The enzyme used to facilitate this transfer is produced by means of fermentation using non-GMO (non-genetically modified organism) bacteria.

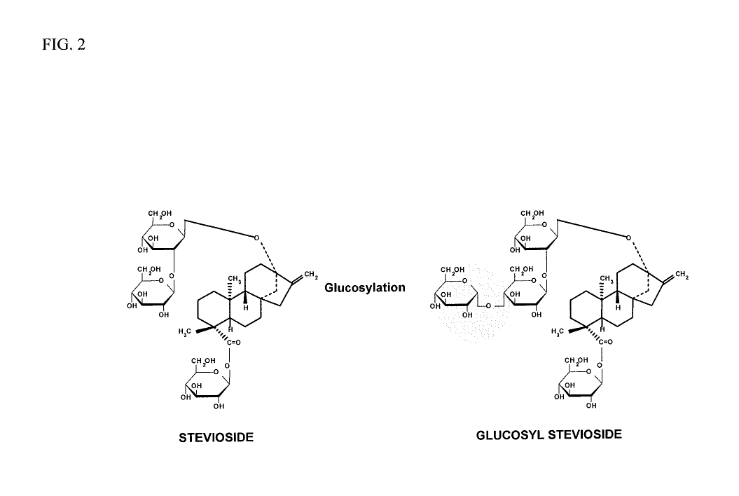

FIG. 2 is an illustration of an example of glucosylation. Specifically, FIG. 2 illustrates the single glucosylation (G1) of a stevioside molecule. This process can yield multiple glucosylation (G2, G3, etc.) of different steviol glycosides (mainly stevioside and Rebaudioside A) present in stevia extract.

TABLE 3

| | | | | Glucosylated Steviol | | |
|---|---|---|---|---|---|---|
| | | | Non-glucosylated | Glycosides | | |
| Test | Molecular | Steviol | Steviol | G1 & | | |
| Ingredients | Wt (avg) | equivalent | Glycosides | G2 | G3-G9 | G10-G20 |
| Stevioside | 805 | 0.396 | >99% | | | |
| Reb A | 967 | 0.329 | >99% | | | |
| Reb D | 1129 | 0.282 | >99% | | | |
| GSG-S | 1210 | 0.263 | <10% | 95% | | |
| GSG-M | 1380 | 0.231 | 7.80% | 42% | 50% | |
| GSG-L | 1798 | 0.177 | 5% | 19% | 60% | 16% |

To evaluate the sweetness potency of various concentrations of stevia products in aqueous solutions, aqueous solutions of sugar, stevioside, Rebaudioside A (Reb A), Rebaudioside D (Reb D), GSG-S (contains mainly smaller GSGs with 1 or 2 glucose units added to SG), GSG-M and GSG-L at various concentrations were prepared using bottled water. Samples were evaluated by the judges at room temperature (70-72° F.).

The judges were 11 panelists that have been previously qualified for their taste acuity and trained in the use of a sweetness intensity rating scale. The evaluations were done in duplicate using the same panelists so that a total of 22 values were generated for each average data point. Prior to the conduct of the study, judges were trained with sugar solutions and the use of the ballot.

Samples were given to the judges sequentially and coded with triple digit numbers. The order of sample presentation was randomized to avoid order of presentation bias. A rest period of five minutes was provided between samples. Water and unsalted crackers were provided in order to cleanse the palate.

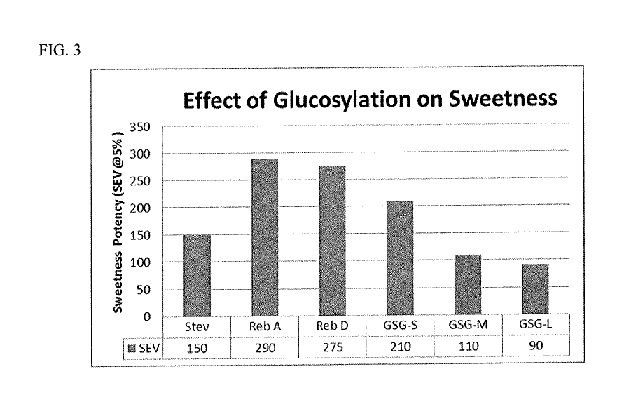

Results were statistically analyzed to generate a standard error value for each solution as well as a confidence level at a 95% level. By comparing the sweetness of each test ingredient to the sweetness of several sucrose solutions, the sweetness potency of different stevia ingredients was estimated as shown in FIG. 3. FIG. 3 is a graph of the sweetness potency, or sucrose equivalent value (SEV), of different stevia ingredients at a 5% sugar sweetness level (i.e. at a concentration equivalent to 5% sucrose). This figure shows the effect of glucosylation on the SEV of steviol glycosides. As the number of glucose units on steviol glycosides increases, the sweetness increases from stevioside to Reb A and then starts decreasing with additional glucose units.

### Example 2

### Effect of Glucosylation on Flavor Modification

A mango-passion fruit flavored water formula was developed to evaluate the effect of different stevia ingredients on the sweetness and flavor profile of the beverage. A total of 9-10 panel members participated in this sensory test, where they assigned relative values to sweetness, onset of sweetness, mango fruit flavor, passion fruit flavor, acidity, overall taste, etc.

### Example 2A

### Effect of Glucosylation on Flavor Modification of No-Sugar-Added Beverage

Table 4 shows the no-sugar added beverage formula that used mainly Reb A, Reb D, GSG-S, or GSG-L. The amount of each ingredient (Reb A: 150 ppm; Reb D: 165 ppm; GSG-S: 190 ppm; and GSG-L: 300 ppm) was selected to have around 50 ppm of steviol in each formula.

TABLE 4
Mango-Passion Fruit Flavored Beverage (No Sugar Added)

| Stevia Ingredient | Reb A | Reb D | GSG-S | GSG-L |
|---|---|---|---|---|
| Water | 95.82 | 95.82 | 95.82 | 95.82 |
| SG Content | 0.015 | 0.0165 | 0.019 | 0.03 |
| Citric Acid | 0.078 | 0.078 | 0.078 | 0.078 |
| Sodium Citrate | 0.056 | 0.056 | 0.056 | 0.056 |
| Mango flavor | 0.031 | 0.031 | 0.031 | 0.031 |
| Passion fruit flavor | 0.014 | 0.014 | 0.014 | 0.014 |

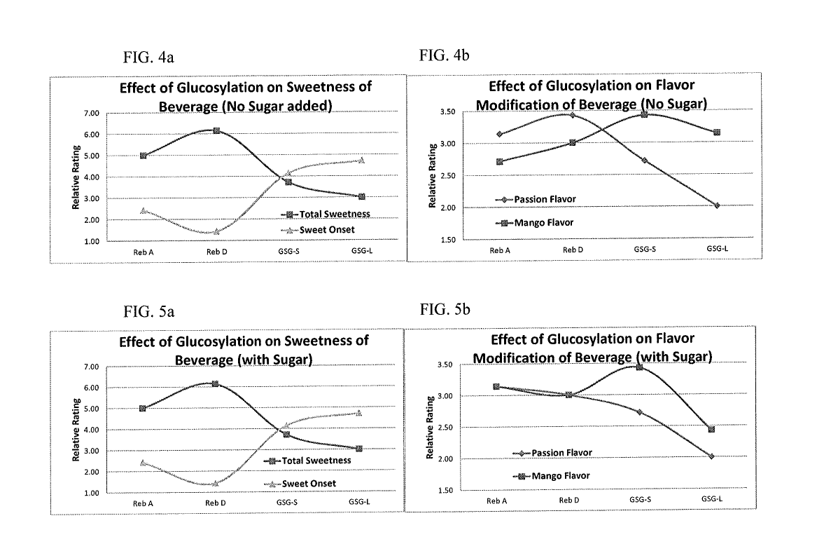

FIGS. 4a-4b show the modification of flavor and sweetness profiles caused by glucosylation. The sweetness intensity decreased and sweet onset delayed with glucosylation. Mango flavor was enhanced and the passion fruit flavor reduced with glucosylation.

### Example 2B

### Effect of Glucosylation on Flavor Modification of a Beverage with Sugar

Table 5 shows the same mango-passion fruit flavored beverage formula with 4% sugar and stevia ingredients that contribute an additional 4% sugar-equivalent sweetness. The formula used Reb A, Reb D, GSG-S, or GSG-L in the amount of 50, 55, 73, and 200 ppm respectively as outlined in the formula below. As the number of glucose units increased more flavor modification (whether enhancement or suppression) was observed as shown in FIGS. 5a-5b.

TABLE 5
Mango-Passion Fruit Flavored Beverage (Sugar Added)

| | Reb A | Reb D | GSG-S | GSG-L |
|---|---|---|---|---|
| Stevia Ingredient | (%) | (%) | (%) | (%) |
| Water | 95.82 | 95.82 | 95.82 | 95.82 |
| Sugar | 4.00 | 4.00 | 4.00 | 4.00 |
| SG Content | 0.005 | 0.0055 | 0.0073 | 0.02 |
| Citric Acid | 0.078 | 0.078 | 0.078 | 0.078 |
| Sodium Citrate | 0.056 | 0.056 | 0.056 | 0.056 |
| Mango flavor | 0.031 | 0.031 | 0.031 | 0.031 |
| Passion fruit flavor | 0.014 | 0.014 | 0.014 | 0.014 |

FIGS. 5a-5b show the effect of glucosylation of SG on the modification of flavor and sweetness profiles of a beverage sweetened with sugar and stevia (SG). The sweetness intensity decreased and sweet onset delayed with glucosylation. Both Mango and passion fruit flavors were somewhat suppressed with glucosylation. A key point is that GSG modified the flavor profile.

### Example 3

### Juice Drink with Glucosyl Steviol Glycosides

In this and the following examples, the GSG may be any glucosyl steviol glycoside composition, such as, but not limited to, a combination of GSG-S, GSG-M, and GSG-L.

To evaluate the flavor modification in a juice drink, a range of GSG concentrations (0 to 1000 ppm) was used with a typical apple blueberry juice drink formula. The objective was to assess whether the addition of GSG has an effect on key flavor attributes in various beverage applications. Specifically, the objective was to determine whether the flavor profile and overall acceptance of a Control sample of apple blueberry juice (containing no GSG) differs from a 30% reduced sugar Test sample of the same beverage (containing GSG). After preliminary sensory tests, it was apparent that GSG modified the flavor and sweetness profile at all concentrations. The GSG concentration is preferably in the range of about 0 to 1000 ppm, more preferably in the range of about 50 to 750 ppm, and most preferably in the range of about 50 to 500 ppm.

For detailed sensory tests, two samples were selected to test against the control sample. The methodology of the detailed sensory tests is set forth below in Table 6. The formulas of the control and test samples are set forth below in Table 7.

TABLE 6
Methodology

| | |
|---|---|
| Number of Sessions | 1 |
| Number of Participants: | 20 |
| Test Design: | Balanced, randomized within pair. Blind |
| Sensory Test Method: | Intensity and acceptance ratings |
| Environmental Condition | Standard booth lighting |
| Attributes and Scales: | |

Overall Acceptance on a 6-pt hedonic scale where 5 = Like Extremely,
2 = Neither Like Nor Dislike, and 0 = Dislike Extremely
Overall Flavor, Apple Flavor, Berry Flavor, Sweetness, Tartness,
Bitterness and Lingering Sweet Aftertaste Intensity on a 6-pt continuous
intensity scale where 0 = Imperceptible and 5 = Extremely Pronounced
Open Ended General Comments

| | |
|---|---|
| Serving Temperature | Refrigerated temperature (~45° F.) |
| Serving/Panelists Instruction: | Samples served simultaneously. Panelists |
| | instructed to read ingredient statement, |
| | evaluate each sample. |

TABLE 7
Samples

| | | Test-1 | Test-2 |
|---|---|---|---|
| Description Flavor | Control | Low GSG Level | Hi GSG Level |
| Apple Juice Concentrate | 6.21% | 6.21% | 6.21% |
| Blueberry Juice | 0.76% | 0.76% | 0.76% |
| Concentrate | | | |
| Apple and blueberry flavor | 0.12% | 0.12% | 0.12% |
| Sugar | 5.60% | 5.60% | 5.60% |
| GSG | | 0.025% | 0.05% |
| Water | Balance | Balance | Balance |

In this study, twenty consumer panel members evaluated three samples of apple blueberry flavored juice drink for overall acceptance and attribute intensities of apple and berry flavors, onset of flavor, sweetness, and aftertaste (includes tartness, bitterness and lingering sweet aftertaste intensity). The three samples included a full sugar control sample containing no Glucosyl Steviol Glycosides (GSG) and two test samples containing low (0.025%) and high (0.05%) levels of GSG. The objective of the test was to determine if the addition of Glucosyl Steviol Glycosides affects the flavor profile of a juice drink. The results indicated:
The Test samples had significantly higher overall acceptability and more apple flavor intensity than the Control sample (at >90% confidence).
The sweetness intensity of the Test sample with low GSG was not significantly different from the control. GSG enhances sweetness and flavor at high level of usage (p=0.047).
There was no significant difference in aftertaste intensities between the test and control samples (at 90% confidence).

### Example 4

### Flavored Carbonated Soft Drink with Glucosyl Steviol Glycosides

To evaluate the flavor modification in a carbonated soft drink, a range of GSG concentrations (0 to 1000 ppm) was used with an orange-pineapple flavored carbonated soft drink formula. The objective was to assess whether the addition of GSG has an effect on key flavor attributes in various carbonated soft drink (CSD) beverage applications. Specifically, the objective was to determine if the flavor profile and overall acceptance of a Control sample of orange pineapple carbonated drink differs from Test samples of the same beverage containing GSG. After preliminary sensory tests, it was apparent that GSG modified the flavor and sweetness profile at all concentrations. The GSG concentration is preferably in the range of about 0 to 1000 ppm, more preferably in the range of about 25 to 750 ppm, and most preferably in the range of about 50 to 500 ppm.

For detailed sensory tests, two samples were selected to test against the control sample. The methodology of the detailed sensory tests is set forth below in Table 8. The formulas of the control and test samples are set forth below in Table 9.

TABLE 8
Methodology

| | |
|---|---|
| Number of Sessions | 1 |
| Number of Participants: | 24 |
| Test Design: | Balanced, randomized within pair. Blind. |
| Sensory Test Method: | Intensity and acceptance ratings |
| Environmental Condition | Standard booth lighting |
| Attributes and Scales: | |

Overall Acceptance on a 6-pt hedonic scale where 5 = Like and
0 = Dislike
Overall Flavor, Orange Flavor, Pineapple Flavor, Aftertaste, and
Sweetness 6-pt continuous intensity scale where 0 = Imperceptible and
5 = Extremely Pronounced

| | |
|---|---|
| Statistical Analysis: | ANOVA (by Block) with Post Hoc |
| | Duncan's Test |
| Sample Size | ~1.5 oz. in a clear capped plastic cup |
| Serving Temperature | Refrigerated temperature (~45° F.) |
| Serving/Panelists Instruction: | Samples served simultaneously. Panelists |
| | instructed to read ingredient statement, |
| | evaluate each sample. |

TABLE 9
Samples

| Ingredients | Control (%) | Test 1 (%) | Test 2 (%) |
|---|---|---|---|
| Water | 90.8 | 90.8 | 90.8 |
| Sugar | 8.93 | 8.93 | 8.93 |
| Acids | 0.142 | 0.142 | 0.142 |
| Orange Flavor | 0.069 | 0.069 | 0.069 |
| Pineapple Flavor | 0.050 | 0.050 | 0.050 |
| SG95 | 0.006 | 0.006 | 0.006 |
| GSG | | 0.025 | 0.05 |

In this study, twenty-four consumer panel members evaluated three samples of orange pineapple fruit flavored carbonated drink for overall acceptance and attribute intensities (overall flavor, orange flavor, pineapple flavor, sweetness, and aftertaste). The three samples included: a reduced sugar control sample containing SG95 (a stevia extract) and two test samples that were the same as the Control sample plus GSG added at 0.025% (low) and 0.05% (High) levels. SG95 is a high purity stevia sweetener available from PureCircle, 915 Harger Road, Suite 250, Oak Brook, Ill. 60523, USA. The objective of the test was to determine if the addition of stevia extract solids affects the flavor profile of a reduced sugar carbonated drink. The results indicated:
The Test sample with low GSG had significantly more orange flavor intensity than the Control sample (95% confidence). The Test sample with high GSG also displayed more orange flavor intensity than the Control sample (p=0.089).
There was no significant difference in overall acceptance, pineapple flavor intensity, or aftertaste intensity between the control and two test samples (at 90% confidence).

### Example 5

### Flavored Milk Drink with Glucosyl Steviol Glycosides

To evaluate the flavor modification in a flavored dairy beverage, a range of GSG concentrations (0 to 1000 ppm) was used with a banana flavored beverage formula. The objective was to determine if the addition of Glucosyl Steviol Glycosides (GSG) has an effect on key flavor attributes and/or improves flavor perception in various beverage applications, specifically in dairy beverages. Specifically, the objective was to determine if the flavor profile and overall acceptance of a Control sample of banana flavored milk drink (containing no GSG) differs from two Test samples of the same drink containing two levels of GSG. Though the test was conducted with banana flavor, the findings are also pertinent with all fruit, vegetable, chocolate, coco flavored beverages and energy drinks. After preliminary sensory tests, it was apparent that GSG modified the flavor and sweetness profile at all concentrations. The GSG concentration is preferably in the range of about 0 to 1000 ppm, more preferably in the range of about 25 to 750 ppm, and most preferably in the range of about 50 to 500 ppm.

For detailed sensory tests, two samples were selected to test against the control sample. The methodology of the detailed sensory tests is set forth below in Table 10. The formulas of the control and test samples are set forth below in Table 11.

TABLE 10
Methodology

| | |
|---|---|
| Number of Sessions | 1 |
| Number of Participants: | 32 |
| Test Design: | Balanced, randomized within pair. Blind. |
| Sensory Test Method: | Intensity and acceptance ratings |
| Environmental Condition | Standard booth lighting |
| Attributes and Scales: | |

Overall Acceptance on a 9-pt hedonic scale where 9 = Like Extremely,
5 = Neither Like Nor Dislike, and 1 = Dislike Extremely
Overall Flavor, Banana Flavor, Dairy Flavor, Sweetness, Tartness
and Bitterness Intensity on a 11-pt continuous intensity scale where
0 = Imperceptible and 10 = Extremely Pronounced

| | |
|---|---|
| Statistical Analysis: | ANOVA (by Block) with Post Hoc |
| | Duncan's Test |
| Sample Size | ~2.0 oz. in a clear capped 3.5 oz. plastic |
| | drinking cup |
| Serving Temperature | Refrigerated temperature (~45° F.) |
| Serving/Panelists Instruction: | Samples served simultaneously. Panelists |
| | instructed to read ingredient statement, |
| | evaluate each sample. |

TABLE 11
Samples
Control: Sugar and Stevia Extract (Reb A)
Test: Sugar, Stevia Extract (Reb A) & GSG

| | | Test-1 | Test-2 |
|---|---|---|---|
| Ingredients | Control | Low GSG Level | High GSG Level |
| MILK (2% fat) | 95.6% | 95.6% | 95.6% |
| SUGAR | 4.2 | 4.2 | 4.2 |
| BANANA FLV | 0.20 | 0.20 | 0.20 |
| Reb A | 0.006 | 0.006 | 0.006 |
| GSG | | 0.0175 | 0.035 |
| TOTAL | 100 | 100 | 100 |

In this study, thirty-two consumer panel members evaluated three samples of reduced sugar banana flavored milk drink for overall acceptance and attribute intensities (overall flavor, banana flavor, dairy flavor, sweetness, and aftertaste intensity). The three samples included: a control sample sweetened with sugar and stevia extract (Reb A) and containing no Glucosyl Steviol Glycosides (GSG) and two test samples sweetened with sugar and stevia extract (Reb A) containing Glucosyl Steviol Glycosides (GSG). One of the test samples contained a low (0.0175%) amount of GSG and the other one contained a high (0.035%) amount of GSG. The objective of the test was to determine if the addition of Glucosyl Steviol Glycosides affects/improves the flavor profile of a banana flavored milk drink. The results indicated:
The Test sample containing a high level of GSG had significantly more banana flavor intensity, sweetness intensity, and delayed onset of dairy flavor note than the Control sample (at 95% confidence).
The test sample containing a low level of GSG also contributed to higher banana flavor intensity (at >90% confidence).
There was no significant difference in overall acceptance, dairy flavor intensity, onset of banana flavor and aftertaste between the test samples and control.

### Example 6

### Baked Goods with Glucosyl Steviol Glycosides

To evaluate the flavor modification in baked goods, a range of GSG concentration (0 to 5000 ppm) was used with a lemon poppy seed muffin formula. The objective was to determine if the addition of Glucosyl Steviol Glycosides has an effect on key flavor attributes and/or improves flavor perception in various food applications, specifically in various baked goods. Specifically, the objective was to determine if the flavor profile and overall acceptance of a Control sample of lemon poppy seed muffin (containing no Glucosyl Steviol Glycosides) differs from a Test sample of the same muffin (containing Glucosyl Steviol Glycosides). Though the test was conducted with muffins, the findings are also pertinent with all baked goods, not limited to cookies, cakes, pastries, bread, etc. After preliminary sensory tests, it was apparent that GSG modified the flavor and sweetness profile at all concentrations. The GSG concentration is preferably in the range of about 0 to 5000 ppm, more preferably in the range of about 100 to 3000 ppm, and most preferably in the range of about 100 to 2000 ppm.

For detailed sensory tests, two samples were selected to test against the control sample. The methodology of the detailed sensory tests is set forth below in Table 12. The formulas of the control and test samples are set forth below in Table 13.

TABLE 12
Methodology

| | |
|---|---|
| Number of Sessions | 1 |
| Number of | 35 |
| Participants: | |
| Test Design: | Balanced, randomized within pair. Blind. |
| Sensory Test Method: | Intensity and acceptance ratings |
| Environmental | Standard booth lighting |
| Condition | |
| Attributes and Scales: | |

Overall Acceptance on a 9-pt hedonic scale where 9 =
Like Extremely, 5 = Neither Like Nor Dislike, and
1 = Dislike Extremely
Overall Flavor, Lemon Flavor, Sweetness, Tartness and
Bitterness Intensity on a 11-pt continuous intensity scale
where 0 = Imperceptible and 10 = Extremely Pronounced
Open Ended General Comments
Gender and Age

| | |
|---|---|
| Statistical Analysis: | ANOVA (by Block) with Post Hoc Duncan's Test |
| Sample Size | ¼ muffin (~14 grams) in a 3.5 oz. plastic soufflé |
| | cup |
| Serving Temperature | ambient temperature (~68° F.) |
| Serving/Panelists | Samples served simultaneously. Panelists |
| Instruction: | instructed to read ingredient statement, evaluate |
| | each sample. |

TABLE 13
Samples

| Ingredients | Control (%) | Test (%) |
|---|---|---|
| AP Flour | 24.37 | 24.35 |
| Milk | 24.01 | 23.99 |
| Sugar | 23.16 | 23.14 |
| Vegetable Oil | 14.79 | 14.78 |
| Eggs | 9.54 | 9.53 |
| Poppy Seeds | 1.00 | 1.00 |
| Lemon Flavor | 0.83 | 0.83 |
| Salt | 0.72 | 0.71 |
| Lemon Juice | 0.59 | 0.59 |
| Vanilla Extract | 0.52 | 0.52 |
| Baking Powder | 0.48 | 0.48 |
| GSG | | 0.07 |
| TOTAL | 100.00 | 100.00 |

In this study, thirty-five consumer panel members evaluated two samples of lemon poppy seed muffins for overall acceptance and attribute intensities (overall flavor, lemon flavor, sweetness, tartness and bitterness intensity). The two samples included: 1) a Control sample containing no Glucosyl Steviol Glycosides (GSG) and 2) a Test sample containing GSG. The objective of the test was to determine if the addition of Glucosyl Steviol Glycosides affects/improves the flavor profile of a lemon poppy seed muffin. The results indicated:
The Test sample (containing Glucosyl Steviol Glycosides) had significantly more overall flavor intensity and sweetness intensity than the Control (at 90% confidence).
There was no significant difference in overall flavor acceptance, lemon flavor intensity, tartness intensity or bitterness intensity between the two samples (at 90% confidence). Directionally, the Test sample was more acceptable overall and had more lemon flavor intensity than the Control (p values=0.124 and 0.190 respectively).
Based on panelist comments, the Control sample had less lemon flavor than the Test sample.

### Example 7

### Spreads & Fruit/Vegetable Preparations with Glucosyl Steviol Glycosides

To evaluate the flavor modification in fruit/vegetable spreads and fruit preparations, a range of GSG concentration (0 to 5000 ppm) was used with a strawberry topping formula for spread. The objective was to determine if the addition of Glucosyl Steviol Glycosides has an effect on key flavor attributes and/or improves flavor perception in various food applications, specifically in fruit or vegetable preparations. Specifically, the objective was to determine if the flavor profile and overall acceptance of a Control sample of strawberry topping (containing no GSG) differs from a Test sample of the same topping (containing GSG). Though the test was conducted with strawberry spread/fruit prep/topping, the findings are also pertinent with fruit and vegetable preparations, not limited to fruits (banana, all berries, mango, etc.) and vegetables (celery, artichoke, squash, avocado, etc). After preliminary sensory tests, it was apparent that GSG modified the flavor and sweetness profile at all concentrations. The GSG concentration is preferably in the range of about 0 to 5000 ppm, more preferably in the range of about 1000 to 4000 ppm, and most preferably in the range of about 2000 to 3000 ppm.

For detailed sensory tests, two samples were selected to test against the control sample. The methodology of the detailed sensory tests is set forth below in Table 14. The formulas of the control and test samples are set forth below in Table 15.

TABLE 14
Methodology

| | |
|---|---|
| Number of Sessions | 1 |
| Number of Participants: | 28 |
| Test Design: | Balanced, randomized within pair. Blind. |
| Sensory Test Method: | Intensity and acceptance ratings |
| Environmental Condition | Standard booth lighting |
| Attributes and Scales: | |

Overall Acceptance on a 9-pt hedonic scale where 9 = Like Extremely,
5 = Neither Like Nor Dislike, and 1 = Dislike Extremely
Overall Flavor, Fresh Strawberry Flavor, Sweetness, Tartness and
Bitterness Intensity on a 11-pt continuous intensity scale where
0 = Imperceptible and 10 = Extremely Pronounced
Open Ended General Comments
Gender and Age

| | |
|---|---|
| Statistical Analysis: | ANOVA (by Block) with Post Hoc |
| | Duncan's Test |
| Sample Size | ~0.5 oz. in a clear capped 1 oz. plastic cup |
| Serving Temperature | Refrigerated temperature (~45° F.) |
| Serving/Panelists Instruction: | Samples served simultaneously. Panelists |
| | instructed to read ingredient statement, |
| | evaluate each sample. |

TABLE 15
Samples

| Ingredients | Control (%) | Test (%) |
|---|---|---|
| Sugar | 39.23 | 39.18 |
| Water | 29.43 | 29.39 |
| Diced Strawberries | 15.44 | 15.42 |
| Strawberry Juice | 15.44 | 15.42 |
| Pectin | 0.26 | 0.26 |
| Potassium Benzoate | 0.10 | 0.10 |
| RebA | 0.06 | 0.06 |
| Citric Acid | 0.05 | 0.05 |
| GSG | | 0.022 |
| TOTAL | 100.00 | 100.00 |

In this study, twenty-eight consumer panel members evaluated two samples of strawberry flavored reduced sugar topping for overall acceptance and attribute intensities (overall flavor, fresh strawberry flavor, sweetness, tartness and bitterness intensity). The two samples included: 1) a control sample sweetened with sugar and Rebaudioside A containing no Glucosyl Steviol Glycosides (GSG) and 2) a test sample sweetened with sugar and Rebaudioside A containing GSG. The objective of the test was to determine if the addition of GSG affects/improves the flavor profile of strawberry flavored topping. The results indicated:
There was no significant difference in overall flavor acceptance, overall flavor intensity, fresh strawberry flavor intensity, sweetness intensity or tartness intensity (at 90% confidence).
The test sample was significantly less bitter than the control (at 90% confidence).

### Example 8

### Yogurt with GSG

To evaluate the flavor modification in flavored and unflavored yogurt, a range of GSG concentration (0 to 1000 ppm) was used with a vanilla flavored yogurt bought from a local store. Though the test was conducted with vanilla flavored yogurt, the findings are also pertinent with all other unflavored and flavored fermented dairy products like cheese, yogurt (with fat or no-fat), drinkable yogurts, smoothies, yogurt with fruit preparations not limited to fruit (banana, all berries, mango, etc.). After preliminary sensory tests, it was apparent that GSG modified the flavor and sweetness profile at all concentrations. The GSG concentration is preferably in the range of about 0 to 1000 ppm, more preferably in the range of about 50 to 500 ppm, and most preferably in the range of about 100 to 400 ppm.

Three vanilla-flavored yogurt samples (generic brand) were sweetened with sugar, sugar+Reb A and Sugar+Reb A+GSG. The amount of GSG was 220 ppm. The twelve member panel found that GSG enhanced the sweetness profile and positively impacted the flavor profile. GSG helped in rounding the sweetness profile and directionally helped in reducing the aftertaste of Reb A.

### Example 9

### Lemon-Lime Carbonated Soft Drink with GSG

To evaluate the flavor modification in a flavored carbonated soft drink (CSD), a range of GSG concentration (0 to 1000 ppm) was used with a lemon-lime flavored CSD. Though the test was conducted with reduced sugar lemon-lime flavored CSD, the findings are also pertinent with all other flavored CSDs (cola, orange, grape fruit, passion fruit, berries fruit group, mango, etc.) with all levels of sugar and diet (no sugar) products. After preliminary sensory tests, it was apparent that GSG modifies the flavor and sweetness profile at all concentrations. The GSG concentration is preferably in the range of about 0 to 1000 ppm, more preferably in the range of about 100 to 500 ppm, and most preferably in the range of about 200 to 400 ppm.

Three CSD samples were made with sugar, Sugar+Reb A, and Sugar+Reb A+GSG. The amount of GSG was 310 ppm. The sample with GSG had directionally improved sweetness profile, enhanced lemon flavor note and reduced bitterness and aftertaste compared to the sample sweetened with sugar and Reb A only.

### Example 10

### Chocolate Milk with GSG

To evaluate the flavor modification in flavored dairy beverages, a range of GSG concentration (0 to 1000 ppm) was used with chocolate milk formulations sweetened with sugar and/or high fructose corn syrup (HFCS) and stevia. Though the test was conducted with a reduced sugar chocolate flavored dairy beverage, the findings are also pertinent with all other flavored dairy beverages with different levels of fat or no-fat with different flavors (strawberry, blueberry, mango, etc.) with different levels of sugar including diet (no sugar) products. After preliminary sensory tests, it was apparent that GSG modifies the flavor and sweetness profile at all concentrations. The GSG concentration is preferably in the range of about 0 to 1000 ppm, more preferably in the range of about 25 to 500 ppm, and most preferably in the range of about 50 to 400 ppm.

One of the test samples was sweetened with a mixture of HFCS42, sugar and Reb A; the other test sample also included 337 ppm of GSG. The GSG enhanced the chocolate flavor, dairy note and sweetness profile.

### Example 11

### Baked Good Frosting with GSG

To evaluate the flavor modification in baked good frostings and spreads, a range of GSG concentration (0 to 0.5%) was used with vanilla flavored cake frosting formulations sweetened with sugar and/or high fructose corn syrup (HFCS) and stevia. Though the test was conducted with a reduced sugar frosting, the findings are also pertinent with all other flavored frostings with different levels of fat or no-fat and different flavors (chocolate, strawberry, blueberry, mango, etc.) with different levels of sugar including no-sugar-added products. After preliminary sensory tests, it was apparent that GSG modified the flavor and sweetness profile at all concentrations. The GSG concentration is preferably in the range of about 0 to 0.5%, more preferably in the range of about 0.1 to 0.4%, and most preferably in the range of about 0.2 to 0.3%.

As an example, a typical sensory test was conducted where GSG was added to a reduced sugar cake frosting. The amount of GSG was 0.23%, which enhanced the sweetness profile as more sugar-like as well as promoted the vanilla flavor.

### Example 12

### Snacks with GSG

To evaluate the flavor modification in snacks and cereal/nut products, a range of GSG concentration (0 to 0.5%) was used with cinnamon flavored coated almonds sweetened with sugar and/or high fructose corn syrup (HFCS). Though the test was conducted with full sugar coated nuts, the findings are also pertinent with all other coatings used for snacks, cereal, confectionery with different level of moisture and fat (or no-fat) and different flavors (chocolate, cinnamon, hazelnut, maple, brown-sugar, strawberry, blueberry, mango, etc.) with different levels of sugar including no-sugar-added products. After preliminary sensory tests, it was apparent that GSG modified the flavor and sweetness profile at all concentrations. The GSG concentration is preferably in the range of about 0 to 0.5%, more preferably in the range of about 0.05 to 0.4%, and most preferably in the range of about 0.1 to 0.3%.

As an example, two coated almond snacks were prepared where the test sample had reduced sugar. GSG was added in the amount of 0.19% to the test sample. The GSG provided the rounded sweetness and enhanced cinnamon flavor to the test sample.

GSG may also be added to confectionery formulations in order modify the flavor of confectionery, such as, but not limited to, hard boiled candy, soft textured confectionery, and chocolates.

### Example 13

### Sweetness Synergy of GSG with Sugar

To evaluate the sweetness detection of GSG, a series of samples was made with NSF-02 (Natural Sweet Flavor #2) in acidified water (pH=3.3, pH was adjusted using citric acid). NSF-02 contains GSG and about 15% to 20% dextrin, and is available from PureCircle, 915 Harger Road, Suite 250, Oak Brook, Ill. 60523, USA.

Aqueous solutions of NSF-02 at various concentrations were prepared using bottled water that was acidified to a pH of about 3.5. The pH was adjusted using 1% citric acid solution within a narrow range. The concentrations of NSF-02 ranged between 0 to 1000 ppm. Samples were evaluated by the judges at room temperature (70-72° F.).

The judges were 10 panelists that have been previously qualified for their taste acuity and trained in the use of a sweetness intensity rating scale. The evaluations were done in duplicate using the same panelists (N=20). Prior to the conduct of the study, judges were presented with sugar controls prepared with the acidified water for the intensity rating on the ballot referencing 2, 4, 6 and 8 on the evaluation scale. These solutions were provided to the judges in order to refresh the judge's memory with the intensity ratings.

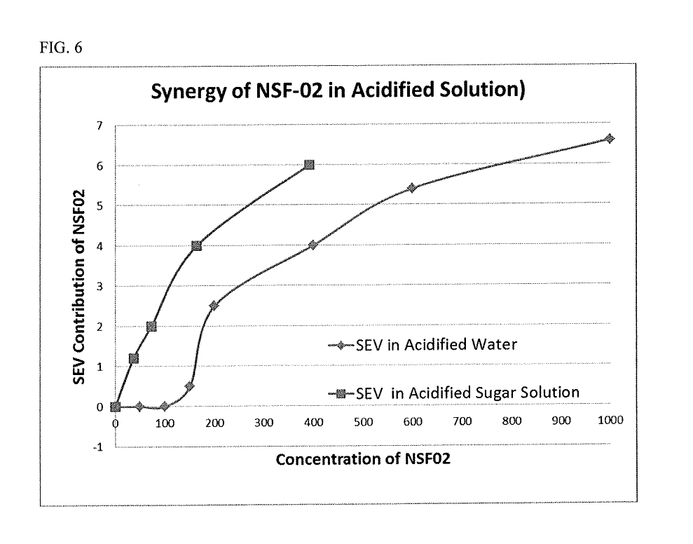

Samples were given to the judges sequentially and coded with triple digit numbers. The order of sample presentation was randomized to avoid order of presentation bias. A rest period of five minutes was provided between samples. Water and unsalted crackers were provided in order to cleanse the palate. The judges could not detect any sweetness below 150 ppm of NSF-02. The sweetness equivalence value (SEV) of different solutions of NSF-02 is shown in FIG. 6.

Similarly, to quantify the synergy between NSF-02 and sugar at 8% sucrose equivalent sweetness in acidified water (about 3.5 pH) at various sugar reduction levels, a sensory test was conducted. The pH was adjusted using 1% citric acid solution within a narrow range. Sugar was reduced by adding the required level of NSF-02 to attain 8% sugar equivalent sweetness. As shown in FIG. 6, the sensory evaluation shows that the addition of NSF-02 contributes additional sweetness in the presence of sugar, even though at a lower level of NSF-02 (0 to 150 ppm of NSF-02 in acidified water, without sugar), it did not contribute any detectable level of sweetness. The synergy between sugar and NSF-02 is the difference in SEV contribution of NSF-02 with and without sugar. The NSF-02 concentration is preferably greater than about 25 ppm, more preferably greater than about 100 ppm, and most preferably in the range of about 500 ppm to 1,000 ppm.

Accordingly, the present invention shows that GSG not only modifies flavor, but it can also enhance sweetness in the presence of other sweeteners. This enhancement of sweetness is caused by the synergy between GSG and other sweeteners.

### Example 14

### GSG Synergy with HFCS

The objective of this experiment was to develop the same sweetness and mouthfeel of High Fructose Corn Syrup (HFCS) 55 in water solution using an equal amount of HFCS 42 plus GSG. HFCS 55 contains a total of 77% dry solid, and 55% of the dry solid is fructose. HFCS 42 contains 71% dry solid, and 42% of the dry solid is fructose. The sweetness equivalence values (SEVs) of HFCS 55 and HFCS 42 are 0.99 and 0.91, respectively.

To match a similar sweetness profile and mouthfeel of HFCS 55, samples were tested including different amounts of GSG from 0 to 500 ppm and different biogums (Xanthan, Gum Arabic, CMC, guar, Locust bean gum, pectin), or polysaccharides (maltodextrin, oligosaccharides, resistant maltodextrin), or polyols. All combinations of GSG and bulking agent (to aid mouthfeel) provided the desired sweetness and mouthfeel that matched HFCS 55. The GSG concentration is preferably in the range of about 0 to 500 ppm, more preferably in the range of about 25 to 300 ppm, and most preferably in the range of about 50 ppm to 200 ppm. However, the best solution was the combination of Xanthan gum and GSG (shown in Table 16). A similar analysis was carried out in a lemon-lime CSD application, which had a combination of GSG and Xanthan gum to provide the similar sweetness and mouthfeel with HFCS 42 as found in the formulation with HFCS 55.

TABLE 16

| Ingredients | HFCS 55 | HFCS 42 |
|---|---|---|
| Water | 87.02 | 87.00 |
| HFCS 55 | 12.98 | — |
| HFCS 42 | — | 12.98 |
| Xanthan Gum | — | 0.0085 |
| GSG | — | 0.0068 |
| Brix | 10 | 9.30 |
| Total | 100 g | 100 g |

It was noticed that when Xanthan gum was added together with GSG the mouth feel perception was improved as well as the overall flavor profile.
The addition of GSG made the sample taste more like a sugar-based product.
   Sensory results:

Sensory analysis (discrimination test) was conducted to determine the difference between the beverage samples with HFCS 55 and HFCS 42. A triangle test was conducted with 19 panelists. Only 4 identified the correct sample, two of whom were guessing. The two that correctly identified the odd sample indicated the differences were due to the HFCS 42 being less acidic and more flavorful; they both also mentioned that HFCS 55 was slightly less sweet than HFCS 42.

Although the tests were conducted using high fructose corn syrups, the results are not limited to high fructose syrups made from corn. The results are also applicable to high fructose syrups made from other carbohydrate sources, such as, but not limited to, wheat, barley, tapioca, rice, and potatoes.

### Example 15

### GSG Synergy with Other Non-Caloric Sweeteners

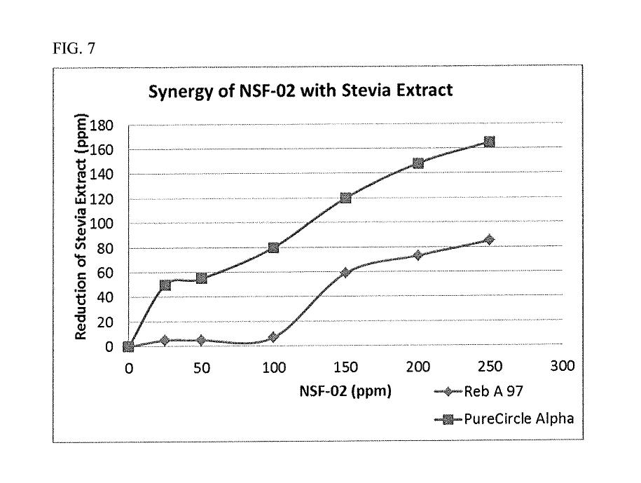

GSG was tested with several natural (Reb A, SG95 and PureCircle Alpha derived from stevia extract) and synthetic sweeteners (Sucralose, Acesulfame-K, cyclamate and aspartame) to investigate the synergy between GSG and high intensity sweeteners. While GSG modifies the flavor profile, it also shows a different degree of synergy with high intensity sweeteners. As an example, to estimate the synergy between GSG and stevia sweeteners, NSF-02 (GSG+dextrin) was mixed with a required quantity of PureCircle Alpha or Reb A 97 in acidic solution (pH=3.8) to attain 8% sugar equivalent sweetness as shown in Table 17. Alpha is a blend of selected steviol glycosides, as described in International Patent Application No. PCT/US2012/024722 filed Feb. 10, 2012, entitled “Stevia Composition,” and is available from PureCircle, 915 Harger Road, Suite 250, Oak Brook, Ill. 60523, USA. Reb A 97 is also available from PureCircle. Synergy was calculated as the reduction of stevia sweeteners (Alpha or Reb A) for the addition of different levels of NSF-02 as shown FIG. 7.

TABLE 17

| | Control | ppm | ppm | ppm | ppm | ppm | ppm |
|---|---|---|---|---|---|---|---|
| NSF-02 | 0 | 25 | 50 | 100 | 150 | 200 | 250 |
| Reb A | 345 | 340 | 340 | 338 | 286 | 272 | 260 |
| Alpha | 400 | 350 | 345 | 320 | 280 | 252 | 235 |
| Synergy with | 0 | 5 | 5 | 7 | 59 | 73 | 85 |
| Reb A | | | | | | | |
| Synergy with | 0 | 50 | 55 | 80 | 120 | 148 | 165 |
| Alpha | | | | | | | |

It was discovered that Alpha shows enhanced sweetness in the presence of a very small amount of NSF-02 (25 ppm or less), whereas more than 100 ppm NSF-02 had to be added to attain any synergy with Reb A as shown in FIG. 7. Note that the detection level of NSF-02 is around 150 ppm as shown in FIG. 6. In a solution with Alpha, the NSF-02 concentration is preferably greater than about 10 ppm, more preferably greater than about 25 ppm, and most preferably greater than about 100 ppm. In a solution with Reb A, the NSF-02 concentration is preferably greater than about 100 ppm, more preferably greater than about 150 ppm, and most preferably greater than about 200 ppm.

### Example 16

### GSG as a Mouthfeel Modifier

GSG works with sugar, HFCS and other natural sweeteners to provide a better mouthfeel and sweetness profile in beverages. Erythritol is used with stevia in beverages to provide some sweetness, but mainly to contribute mouthfeel that is lacking when a high amount of sugar is replaced with high intensity sweetener. A study was conducted to investigate the amount of erythritol that can be replaced with GSG in still beverages.

Sample preparation: A number of acidified beverage samples were targeted to 8 Brix sweetness level with 200 ppm of Reb A and the combination of erythritol and GSG as shown in Table 18.

TABLE 18

| | Erythritol Conc (%) | | | | |
|---|---|---|---|---|---|
| | 3.5 | 2.5 | 1.5 | 3 | 2.75 |
| | E | E6 | E7 | E8 | E9 |
| Reb A | 0.1 | 0.1 | 0.1 | 0.1 | 0.1 |
| GSG | | 0.044 | 0.0875 | 0.022 | 0.033 |
| Erythritol | 17.5 | 12.5 | 7.5 | 15 | 13.75 |
| Citric | 0.25 | 0.25 | 0.25 | 0.25 | 0.25 |
| Water | 482.2 | 487.1 | 492.1 | 484.6 | 485.9 |
| Total | 500 | 500 | 500 | 500 | 500 |

Sensory Evaluation:
1. In a comparison of E, E6 and E7 samples, E6 was found to be very close to control (E) on flavor and overall mouthfeel, and slightly less sweet than control. E7 was very watery.
2. A triangle test with E and E6 samples was conducted over two days with a total of 12 panel members. Five of them detected the correct sample. The conclusion was that panel members could detect the difference; thus the test failed.
3. A preliminary test with E, E8, E9 was then conducted, a triangle test with E and E9 was run. Three out of ten panel members could identify the correct sample.

Conclusion: GSG can reduce erythritol usage by 20-30% (from 3.5% to 2.75-3%) in beverages without any sacrifice of taste or mouthfeel. In the flavor system studied, the GSG concentration is preferably greater than about 10 ppm, more preferably greater than about 20 ppm, and most preferably in the range of about 30 ppm to about 200 ppm. In some flavor systems, GSG may replace more erythritol to provide a balanced, rounded sweetness flavor.

### Example 17

### Effect of Glucosylated Steviol Glycoside on Belgian White Wheat Ale

Flavor modification from glucosylated steviol glycoside (GSG) on an alcoholic beverage was tested using a commercially available Belgian white wheat ale. The two samples (Table 19) included: 1) the unaltered ale as purchased (Control), and 2) the test sample with the addition of 175 ppm glucosylated steviol glycoside. A 30 member panel evaluated the samples described for overall acceptance and attribute intensities (sweet intensity, bitterness, orange flavor, alcohol note, sweet aftertaste, bitter aftertaste). Table 20 describes the testing protocol.

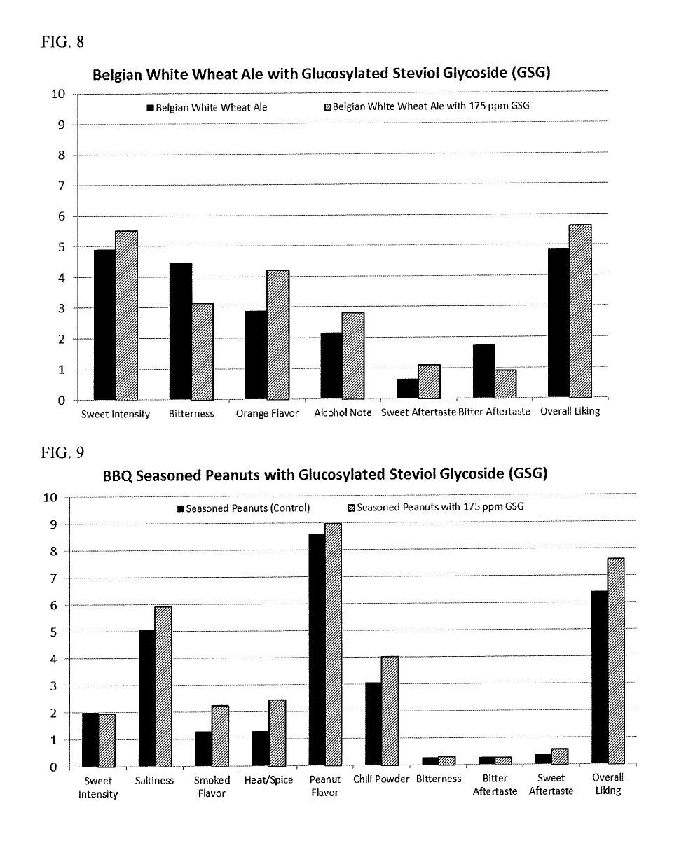

Table 21 shows the sensory results for the control and test products. The test product with 175 ppm glucosylated steviol glycoside was lower in bitterness, higher in orange flavor and higher in sweet aftertaste at 95% confidence, higher in sweet intensity and higher in the alcohol note at 90% confidence, and directionally higher in overall liking (80% confidence). FIG. 8 illustrates this comparison.

TABLE 19
Commercially Produced Belgian White Wheat Ale

| | Market Product | Market Product with |
|---|---|---|
| | (Control) | GSG |
| Belgian White Wheat Ale | 100.00 | 99.9825 |
| Glucosylated Steviol Glycoside | — | 0.0175 |
| Total | 100 | 100 |

TABLE 20
Sensory evaluation of Belgian White Wheat Ale

| | |
|---|---|
| Nature of Participants: | Company employees |
| Number of Sessions | 1 |
| Number of | 15 |
| Participants: | |
| Test Design: | Balanced, randomized within set. Blind |
| Sensory Test | Intensity and acceptance ratings |
| Method: | |
| Environmental | Standard booth lighting |
| Condition | |
| Attributes and Scales: | |

Overall Acceptance on a 10-pt hedonic scale where
10 = Extremely Like and 0 = Extremely Dislike
Sweetness, bitterness, orange flavor, alcohol note, overall
liking and aftertaste. 10-pt continuous intensity scale where
0 = Imperceptible and 10 = Extremely Pronounced

| | |
|---|---|
| Statistical Analysis: | ANOVA (by Block) with Post Hoc Duncan's Test |
| Sample Size | ~1.5 oz. in a clear capped plastic cup |
| Serving Temperature | Refrigerated temperature (~45° F.) |
| Serving/Panelists | Samples served simultaneously. Panelists |
| Instruction: | instructed to evaluate each sample. |

TABLE 21
Results from sensory evaluation of Belgian White Wheat Ale
Summary of the overall acceptance and mean attribute
intensity results for Belgian white wheat ale with
glucosylated steviol glycoside

| | Market Product | Market Product with | | |
|---|---|---|---|---|
| Attribute | (Control) | GSG | p-value | Sig |
| Sweet intensity | 4.89 | 5.50 | 0.0972 | ** |
| Bitterness | 4.45 | 3.13 | 0.0031 | *** |
| Orange flavor | 2.89 | 4.20 | 0.0001 | *** |
| Alcohol note | 2.15 | 2.80 | 0.0774 | ** |
| Sweet aftertaste | 0.62 | 1.08 | 0.0036 | *** |
| Bitter aftertaste | 1.74 | 0.89 | 0.0032 | *** |
| Overall liking | 4.85 | 5.62 | 0.1032 | * |

* = 80% CI,
** = 90% CI,
*** = 95% CI

From this Example, it can be seen that the addition of a glucosylated steviol glycoside flavor modifier ingredient unexpectedly reduced the bitterness and bitter aftertaste in an alcoholic beverage as compared to the control, and improved the alcohol note and other taste and flavor attributes as well as the overall liking as compared to the control. The useful usage level of glucosylated steviol glycoside flavor modifier ingredients ranges from about 125 ppm to about 175 ppm in alcoholic beverages.

### Example 18

### Effect of Glucosylated Steviol Glycosides on BBQ Seasoned Peanuts

A BBQ seasoning for peanuts was developed to determine the impact of glucosylated steviol glycosides on salty snacks. The two samples (Table 22) included: 1) control sample of BBQ seasoned peanuts, and 2) test sample of BBQ seasoned peanuts with 175 ppm glucosylated steviol glycoside. A 30 member panel evaluated both samples for overall acceptance and attribute intensities (sweet intensity, saltiness, smoked flavor, heat/spice, peanut flavor, chili powder, bitterness, bitter aftertaste and sweet aftertaste). Table 23 describes the sensory protocol.

Table 24 shows that the seasoned peanut sample containing GSG had significantly higher smoked flavor, heat/spice, and chili powder intensity at 95% confidence and significantly higher saltiness at 90% confidence. In addition, the test sample GSG had directionally higher peanut flavor, and sweet aftertaste compared to the control at 80% confidence. FIG. 9 illustrates this comparison.

TABLE 22
BBQ Seasoned Peanuts

| | Seasoned Peanuts | Seasoned Peanuts with |
|---|---|---|
| | (Control) | 175 ppm GSG |
| Dry Roasted Unsalted | 93.649 | 93.6315 |
| Peanuts | | |
| Vegetable Oil | 2 | 2 |
| Sodium Chloride | 2 | 2 |
| Cumin Powder | 0.286 | 0.286 |
| Chili Powder | 0.173 | 0.173 |
| Garlic Powder | 0.156 | 0.156 |
| Ground Cayenne Pepper | 0.156 | 0.156 |
| Smoke Flavor | 0.08 | 0.08 |
| Sucrose | 1.5 | 1.5 |
| Glucosylated Steviol | — | 0.0175 |
| Glycoside | | |
| Total | 100 | 100 |

TABLE 23
Sensory evaluation of BBQ seasoned peanuts

| | |
|---|---|
| Nature of Participants: | Company employees |
| Number of Sessions | 1 |
| Number of | 30 |
| Participants: | |
| Test Design: | Balanced, randomized within set. Blind |
| Sensory Test | Intensity and acceptance ratings |
| Method: | |
| Environmental | Standard booth lighting |
| Condition | |
| Attributes and Scales: | |

Overall Acceptance on a 10-pt hedonic scale where
10 = Extremely Like and 0 = Extremely Dislike
Sweetness, saltiness, smoked flavor, heat/spice, peanut flavor,
chili powder, bitterness, and aftertaste. 10-pt continuous intensity
scale where 0 = Imperceptible and 10 = Extremely Pronounced

| | |
|---|---|
| Statistical Analysis: | ANOVA (by Block) with Post Hoc Duncan's Test |
| Sample Size | ~1.5 oz. in a clear capped plastic cup |
| Serving Temperature | Room temperature |
| Serving/Panelists | Samples served simultaneously. Panelists |
| Instruction: | instructed to evaluate each sample. |

TABLE 24
Summary of the overall acceptance and mean attribute intensity
results for BBQ seasoned peanuts with glucosylated steviol
glycoside

| | | Seasoned Peanuts | | |
|---|---|---|---|---|
| | Seasoned Peanuts | with 175 ppm | | |
| Attribute | (Control) | GSG | p-value | Sig |
| Sweet Intensity | 1.98 | 1.94 | 0.8548 | NS |
| Saltiness | 5.05 | 5.92 | 0.0688 | ** |
| Smoked Flavor | 1.28 | 2.23 | 0.0373 | *** |
| Heat/Spice | 1.28 | 2.42 | 0.0207 | *** |
| Peanut Flavor | 8.57 | 8.97 | 0.16 | * |
| Chili Powder | 3.04 | 4.01 | 0.0226 | *** |
| Bitterness | 0.27 | 0.31 | 0.5419 | NS |
| Bitter Aftertaste | 0.28 | 0.26 | 0.6509 | NS |
| Sweet Aftertaste | 0.35 | 0.56 | 0.1673 | * |
| Overall Liking | 6.40 | 7.61 | 0.0058 | *** |

* = 80% CI,
** = 90% CI,
*** = 95% CI

From this Example, it can be seen that the addition of a glucosylated steviol glycoside flavor modifier ingredient unexpectedly enhanced savory attributes such as saltiness, smoked flavor, and heat/spiciness, while having an insignificant impact on bitterness or bitter aftertaste, as compared to the control, and improved the overall liking as compared to the control. The useful usage level of glucosylated steviol glycoside flavor modifier ingredients ranges from about 133 ppm to about 175 ppm in snack foods and nut products.

### Example 19

### Effect of Glucosylated Steviol Glycosides on Ranch Dressing

A ranch dressing was formulated to determine the impact of glucosylated steviol glycosides in a fats and oils application. The two samples (Table 25) included: 1) control sample of ranch dressing, and 2) test sample of ranch dressing with 190 ppm glucosylated steviol glycoside. A 30 member panel evaluated both samples for overall acceptance and attribute intensities (sweetness, bitterness, sourness, dill flavor, garlic, vinegar, creaminess, balanced, sweet aftertaste and bitter aftertaste). Table 26 describes the sensory protocol.

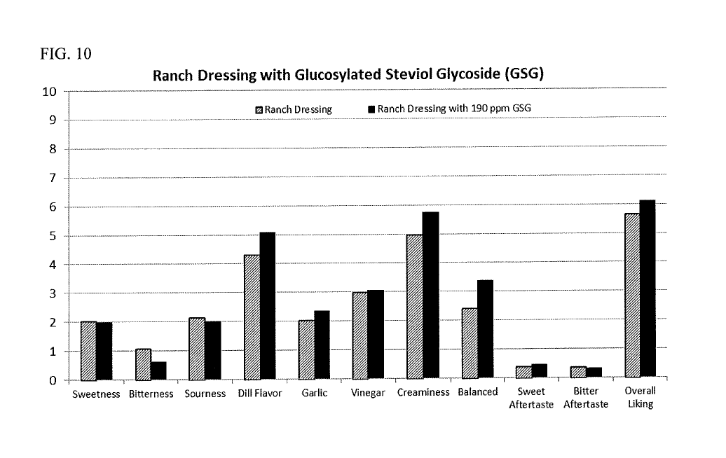

Table 27 shows that the ranch dressing sample containing GSG had significantly lower bitter intensity and significantly higher dill flavor, creaminess, and was more balanced at 95% confidence. In addition, the test sample GSG had directionally higher garlic, and overall liking compared to the control at 80% confidence. FIG. 10 illustrates this comparison.

TABLE 25
Ranch Dressing

| | Ranch Dressing | Ranch Dressing with |
|---|---|---|
| | (Control) | 190 ppm GSG |
| Sour Cream | 38.200 | 38.200 |
| Low Fat Cultured Buttermilk | 38.200 | 38.181 |
| Mayonnaise | 19.100 | 19.100 |
| Minced Dried Garlic | 0.309 | 0.309 |
| Salt | 0.955 | 0.955 |
| Black Pepper | 0.191 | 0.191 |
| Dried Dill Weed | 0.525 | 0.525 |
| Freeze Dried Chives | 0.102 | 0.102 |
| 100% Organic Lemon Juice | 2.418 | 2.418 |
| GSG | — | 0.019 |
| Total | 100 | 100 |

TABLE 26
Sensory evaluation of Ranch Dressing

| | |
|---|---|
| Nature of Participants: | Company employees |
| Number of Sessions | 1 |
| Number of | 30 |
| Participants: | |
| Test Design: | Balanced, randomized within set. Blind |
| Sensory Test | Intensity and acceptance ratings |
| Method: | |
| Environmental | Standard booth lighting |
| Condition | |
| Attributes and Scales: | |

Overall Acceptance on a 10-pt hedonic scale where
10 = Extremely Like and 0 = Extremely Dislike
Sweetness, bitterness, sourness, dill flavor, garlic, vinegar,
creaminess, balanced, and aftertaste 10-pt continuous intensity
scale where 0 = Imperceptible and 10 = Extremely Pronounced

| | |
|---|---|
| Statistical Analysis: | ANOVA (by Block) with Post Hoc Duncan's Test |
| Sample Size | ~1.5 oz. in a clear capped plastic cup |
| Serving Temperature | Refrigerated temperature |
| Serving/Panelists | Samples served simultaneously. Panelists |
| Instruction: | instructed to evaluate each sample. |

TABLE 27
Summary of the overall acceptance and mean attribute intensity
results for ranch dressing with glucosylated steviol glycoside

| | Ranch | Ranch Dressing with | | |
|---|---|---|---|---|
| Attribute | Dressing | 190 ppm GSG | p-value | Sig |
| Sweetness | 2.01 | 1.98 | 0.906 | NS |
| Bitterness | 1.05 | 0.63 | 0.0001 | *** |
| Sourness | 2.13 | 2.01 | 0.5471 | NS |
| Dill Flavor | 4.28 | 5.09 | 0.0163 | *** |
| Garlic | 2.01 | 2.34 | 0.1359 | * |
| Vinegar | 2.96 | 3.06 | 0.6633 | NS |
| Creaminess | 4.94 | 5.75 | 0.0015 | *** |
| Balanced | 2.40 | 3.38 | 0.0066 | *** |
| Sweet Aftertaste | 0.38 | 0.47 | 0.3144 | NS |
| Bitter Aftertaste | 0.35 | 0.32 | 0.6531 | NS |
| Overall Liking | 5.64 | 6.11 | 0.1922 | * |

* = 80% CI,
** = 90% CI,
*** = 95% CI

From this Example, it can be seen that the addition of a glucosylated steviol glycoside flavor modifier ingredient unexpectedly reduced the bitterness and bitter aftertaste in a fat or oil-based consumable product, as compared to the control, and improved the creamy mouthfeel and other taste and flavor attributes as well as the overall liking as compared to the control. The useful usage level of glucosylated steviol glycoside flavor modifier ingredients ranges from about 125 ppm to about 190 ppm in fat- or oil-based food products.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the invention described in the specification. As one of ordinary skill in the art will readily appreciate from the disclosure of the present invention, the compositions, devices, processes, methods, and steps, presently existing or later to be developed that perform substantially the same function or achieve substantially the same result as the corresponding embodiments described herein may be utilized according to the present invention.

What is claimed is:

1. A taste and flavor enhancing composition, comprising a glucosylated steviol glycoside.

2. The taste and flavor enhancing composition of claim 1, wherein the glucosylated steviol glycoside comprises more than two glucose units.

3. The taste and flavor enhancing composition of claim 1, wherein the glucosylated steviol glycoside comprises more than three glucose units.

4. The taste and flavor enhancing composition of claim 2, wherein at least one glucose unit occurs at position C-19 of the steviol glycoside.

5. The taste and flavor enhancing composition of claim 1, further comprising dextrin.

6. A food or beverage product having an intense taste and flavor profile comprising the taste and flavor enhancing composition of claim 1.

7. The food or beverage product of claim 6, selected from the group consisting of a carbonated soft drink, a fruit juice, a dairy food, a dairy beverage, a baked good, a cereal product, and a table top sweetener.

8. The food or beverage product of claim 6, wherein the intense taste and flavor profile is more intense than a comparative taste and flavor profile of a comparative food or beverage product which does not include the taste and flavor enhancing composition.

9. The food or beverage product of claim 6, wherein a mouthfeel of said food or beverage product is improved in relation to a mouthfeel of a comparative food or beverage product which does not include the taste and flavor enhancing composition.

10. A method for making the taste and flavor enhancing composition of claim 1, comprising the steps of:
a. Extracting steviol glycosides from leaves of a Steviol rebaudiana Bertoni plant; and
b. Transglycosylating the steviol glycosides to add glucose units to the steviol glycosides.

11. The method of claim 10, wherein transglycosylating the steviol glycosides comprises enzymatic transglycosylation using an enzyme selected from the group consisting of pullulanase, isomaltase, β-galactosidase, dextrine saccharase, and cyclodextrin glucotransferase.

12. A method of increasing a taste and flavor intensity of a food or beverage product, comprising the step of adding the taste and flavor enhancing composition of claim 1 to the food or beverage product.

13. A method of improving a sweetness of a food or beverage product, comprising adding a high fructose syrup and the taste and flavor enhancing composition of claim 1 to the food or beverage product.

14. The method of claim 13, wherein the high fructose syrup comprises a high fructose corn syrup.

15. The method of claim 14, wherein the high fructose corn syrup comprises high fructose corn syrup 42 (HFCS 42).

16. A food or beverage product having improved sweetness made by the method of claim 13.

17. A method of improving the organoleptic properties of a food or beverage product including a high fructose syrup, comprising the step of adding the taste and flavor enhancing composition of claim 1 to said food or beverage product.

18. The method of claim 17, wherein the high fructose syrup comprises a high fructose corn syrup.

19. The method of claim 18, wherein the high fructose corn syrup comprises high fructose corn syrup 42 (HFCS 42).

20. A food or beverage product having improved organoleptic properties made by the method of claim 17.

21. A method of making a sweetened food or beverage product, comprising the step of adding the taste and flavor enhancing composition of claim 1 and a reduced amount of erythritol to a food or beverage product, wherein said reduced amount of erythritol is less than an amount of erythritol in a comparative food or beverage product sweetened by erythritol which does not include the taste and flavor enhancing composition, and wherein the sweetened food or beverage product has a mouthfeel which is similar to a mouthfeel of the comparative food or beverage product sweetened by erythritol.

22. The method of claim 21, wherein said reduced amount of erythritol is in the range of about 20% to 30% by weight less than the amount of erythritol in the comparative food or beverage product sweetened by erythritol.

23. A sweetened food or beverage product made by the method of claim 21.

24. A method for preparing an alcoholic beverage product comprising adding a glucosylated steviol glycoside flavor modifier ingredient to the beverage, wherein the beverage has improved alcohol note flavor and reduced bitterness and bitter aftertaste compared to a control alcoholic beverage made without the glucosylated steviol glycoside flavor modifier ingredient.

25. The method of claim 24, wherein the flavor modifier ingredient is added at an amount ranging from 125 ppm to about 175 ppm.

26. The method of claim 24, wherein about 175 ppm of the flavor modifier ingredient is added to the alcoholic beverage product.

27. A method for preparing a savory product comprising adding a glucosylated steviol glycoside flavor modifier ingredient to the savory product, wherein the savory product has improved savory attributes compared to a control savory snack product made without the glucosylated steviol glycoside flavor modifier ingredient.

28. The method of claim 27, wherein the flavor modifier ingredient is added at an amount ranging from 133 ppm to about 175 ppm.

29. The method of claim 27, wherein the savory product is a nut product, and about 175 ppm of the flavor modifier ingredient is added to the nut product.

30. A method for preparing a fat-based food product, comprising adding a glucosylated steviol glycoside flavor modifier ingredient to the food product, wherein the food product has improved creamy mouthfeel and reduced bitterness and bitter aftertaste compared to a control food product made without the glucosylated steviol glycoside flavor modifier ingredient.

31. The method of claim 30, wherein the flavor modifier ingredient is added at an amount ranging from 125 ppm to about 190 ppm.

32. The method of claim 30, wherein about 190 ppm of the flavor modifier ingredient is added to the fat-based food product.
